(19) United States
(12) Patent Application Publication
(10) Pub. No.: US 2007/0233156 A1
(43) Pub. Date: Oct. 4, 2007

(54) SURGICAL INSTRUMENT

(76) Inventor: Robert Metzger, Warsaw, IN (US)

Correspondence Address: BOSE MCKINNEY & EVANS LLP;JAMES COLES, 135 N PENNSYLVANIA ST, SUITE 2700, INDIANAPOLIS, IN, 46204, US

(21) Appl. No.: 11/626,976

(22) Filed: Jan. 25, 2007

Related U.S. Application Data
(60) Provisional application No. 60/773,992, filed on Feb. 16, 2006.

(51) Int. Cl. A61B 19/00

(52) U.S. Cl. 606/130

(57) ABSTRACT

An instrument for use with a surgical navigation system to aid in cutting a bone is provided. The instrument includes an anchoring block configured for attachment to a bone and a cutting block or guide having a cutting slot or other guiding surface. The cutting block is also configured for attachment to a bone. A connecting member connects the anchoring block to the cutting block and permits the cutting block to move relative to the anchoring block within a pre-determined range of motion. The connecting member also prevents movement of the cutting block relative to the anchoring block beyond the pre-determined range of motion. In certain embodiments, the connecting member can be configured to provide resistance to movement of the cutting block relative to the anchoring block, such that the cutting block can maintain its position without being held by the physician.

### RELATED APPLICATIONS

This application claims priority to U.S. provisional application Ser. No. 60/773,992, filed Feb. 16, 2006, the entire disclosure of which is hereby incorporated by reference.

### FIELD OF THE INVENTION

The present teachings relate to surgical navigation and more particularly to a method of using surgical navigation to perform cuts to a bone.

### BACKGROUND

Surgical navigation systems, also known as computer assisted surgery and image guided surgery, aid surgeons in locating patient anatomical structures, guiding surgical instruments, and implanting medical devices with a high degree of accuracy. Surgical navigation has been compared to a global positioning system that aids vehicle operators to navigate the earth. A surgical navigation system typically includes a computer, a tracking system, and patient anatomical information. The patient anatomical information can be obtained by using an imaging mode such a fluoroscopy, computer tomography (CT) or by simply defining the location of patient anatomy with the surgical navigation system. Surgical navigation systems can be used for a wide variety of surgeries to improve patient outcomes.

To successfully implant a medical device, surgical navigation systems often employ various forms of computing technology, as well as utilize intelligent instruments, digital touch devices, and advanced 3-D visualization software programs. All of these components enable surgeons to perform a wide variety of standard and minimally invasive surgical procedures and techniques. Moreover, these systems allow surgeons to more accurately plan, track and navigate the placement of instruments and implants relative to a patient's body, as well as conduct pre-operative and intra-operative body imaging.

### SUMMARY OF THE INVENTION

The present teachings provide a cutting block instrument and method of using it with a surgical navigation system.

In one exemplary embodiment, the present teachings provide an instrument for use with a surgical navigation system to aid in cutting a bone. The instrument comprises a first block configured for attachment to a bone and a second block having a guiding surface for a cutting instrument and being configured for attachment to a bone. A connecting member connects the first block to the second block and permits the second block to move relative to the first block within a pre-determined range of motion and prevents movement of the second block relative to the first block beyond the pre-determined range of motion.

In certain exemplary embodiments, the connecting member provides resistance to movement of the second block relative to the first block, such that the second block can maintain its position without a person holding it. In other exemplary embodiments, the second block comprises a distal femur cutting block.

### BRIEF DESCRIPTION OF THE DRAWINGS

The above-mentioned aspects of the present teachings and the manner of obtaining them will become more apparent and the invention itself will be better understood by reference to the following description of the embodiments of the invention taken in conjunction with the accompanying drawings, wherein:

FIG. 1 is a perspective view of an exemplary operating room setup in a surgical navigation embodiment in accordance with the present teachings;

FIG. 2 is an exemplary block diagram of a surgical navigation system embodiment in accordance with the present teachings;

FIG. 3 is an exemplary surgical navigation kit embodiment in accordance with the present teachings;

FIG. 4 is a fragmentary perspective view of components of an instrument and a surgical navigation system in accordance with the present teachings;

FIG. 5 is a fragmentary perspective view of components shown in FIG. 4, with the instrument being shown in a different position;

FIG. 6 is a fragmentary perspective view of an instrument in accordance with the present teachings shown being secured to the femur of a patient;

FIG. 7 is a fragmentary perspective view of an instrument in accordance with the present teachings being used to guide a surgical saw in making a cut to the femur of a patient;

FIG. 8 is a fragmentary perspective view illustrating the femur cut with the surgical saw shown in FIG. 7;

FIGS. 9 and 9A are, respectively, perspective and side views of an alternate embodiment of a surgical instrument in accordance with the present teachings.

FIG. 10 is a perspective view of yet another alternate embodiment of a surgical instrument in accordance with the present teachings; and

FIG. 11 is a perspective view of still another embodiment of a surgical instrument in accordance with the present teachings.

Corresponding reference characters indicate corresponding parts throughout the several views.

### DETAILED DESCRIPTION

The embodiments of the present teachings described below are not intended to be exhaustive or to limit the invention to the precise forms disclosed in the following detailed description. Rather, the embodiments are chosen and described so that others skilled in the art may appreciate and understand the principles and practices of the present teachings.

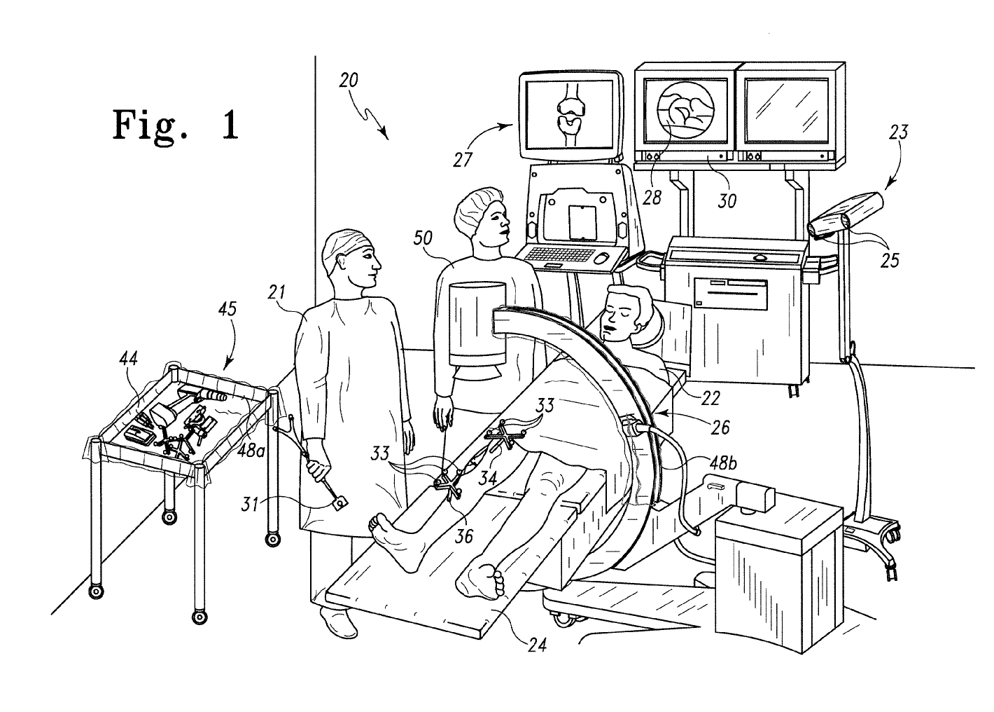

FIG. 1 shows a perspective view of an operating room with surgical navigation system 20. Surgeon 21 is aided by the surgical navigation system in performing knee arthroplasty, also known as knee replacement surgery, on patient 22 shown lying on operating table 24. Surgical navigation system 20 has a tracking system that locates arrays and tracks them in real-time. To accomplish this, the surgical navigation system includes optical locator 23, which has two CCD (charge couple device) cameras 25 that detect the positions of the arrays in space by using triangulation. The relative location of the tracked arrays, including the patient's anatomy, can then be shown on a computer display (such as computer display 27 for instance) to assist the surgeon during the surgical procedure. The arrays that are typically used include probe arrays, instrument arrays, reference arrays, and calibrator arrays. The operating room includes an imaging system such as C-arm fluoroscope 26 with fluoroscope display image 28 to show a real-time image of the patient's knee on monitor 30. The tracking system also detects the location of surgical components, such as spatula probe 31, as well as reference arrays 34, 36, which are attached to the patient's femur and tibia. By knowing the location of markers 33 attached to the surgical components, the tracking system can detect and calculate the position of the components in space. The operating room also includes instrument cart 45 having tray 44 for holding a variety of surgical instruments and arrays 46. Instrument cart 45 and C-arm 26 are typically draped in sterile covers 48a, 48b to eliminate contamination risks within the sterile field.

The surgery is performed within a sterile field, adhering to the principles of asepsis by all scrubbed persons in the operating room. Patient 22, surgeon 21 and assisting clinician 50 are prepared for the sterile field through appropriate scrubbing and clothing. The sterile field will typically extend from operating table 24 upward in the operating room. Typically both the computer display and fluoroscope display are located outside of the sterile field.

A representation of the patient's anatomy can be acquired with an imaging system, a virtual image, a morphed image, or a combination of imaging techniques. The imaging system can be any system capable of producing images that represent the patient's anatomy such as a fluoroscope producing x-ray two-dimensional images, computer tomography (CT) producing a three-dimensional image, magnetic resonance imaging (MRI) producing a three-dimensional image, ultrasound imaging producing a two-dimensional image, and the like. A virtual image of the patient's anatomy can be created by defining anatomical points with surgical navigation system 20 or by applying a statistical anatomical model. A morphed image of the patient's anatomy can be created by combining an image of the patient's anatomy with a data set, such as a virtual image of the patient's anatomy. Some imaging systems, such as C-arm fluoroscope 26, can require calibration. The C-arm can be calibrated with a calibration grid that enables determination of fluoroscope projection parameters for different orientations of the C-arm to reduce distortion. A registration phantom can also be used with a C-arm to coordinate images with the surgical navigation application program and improve scaling through the registration of the C-arm with the surgical navigation system. A more detailed description of a C-arm based navigation system is provided in James B. Stiehl et al., Navigation and Robotics in Total Joint and Spine Surgery, Chapter 3: C-Arm-Based Navigation, Springer-Verlag (2004).

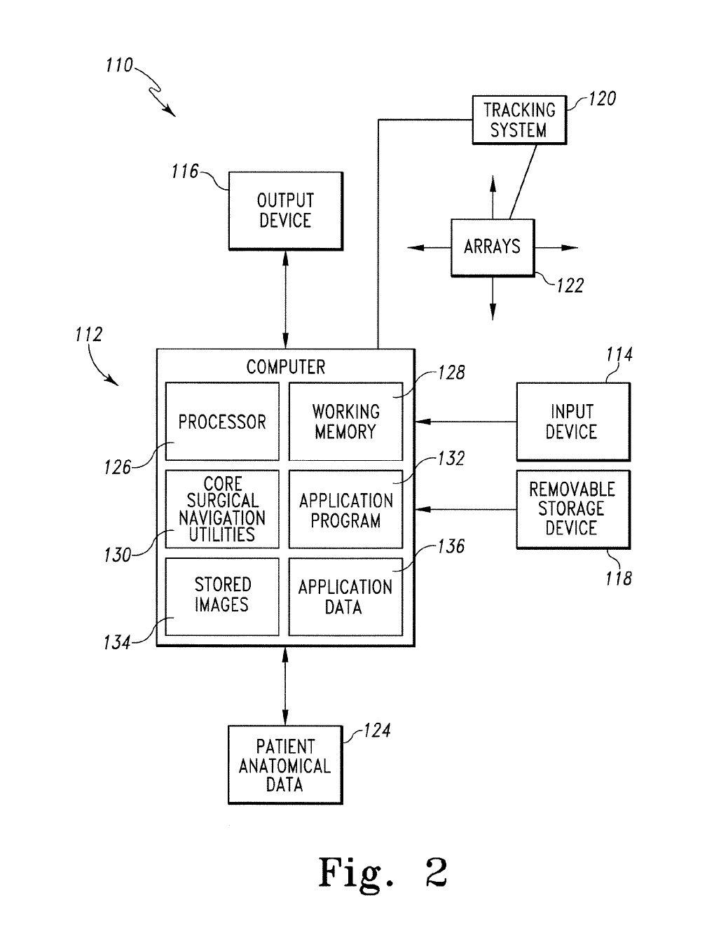

FIG. 2 is a block diagram of an exemplary surgical navigation system embodiment in accordance with the present teachings, such as an Acumen™ Surgical Navigation System, available from EBI, L.P., Parsipanny, N.J. USA, a Biomet Company. The surgical navigation system 110 comprises computer 112, input device 114, output device 116, removable storage device 118, tracking system 120, arrays 122, and patient anatomical data 124, as further described in the brochure Acumen™ Surgical Navigation System, Understanding Surgical Navigation (2003) available from EBI, L.P. The Acumen™ Surgical Navigation System can operate in a variety of imaging modes such as a fluoroscopy mode creating a two-dimensional x-ray image, a computer-tomography (CT) mode creating a three-dimensional image, and an imageless mode creating a virtual image or planes and axes by defining anatomical points of the patient's anatomy. In the imageless mode, a separate imaging device such as a C-arm is not required, thereby simplifying set-up. The Acumen™ Surgical Navigation System can run a variety of orthopedic applications, including applications for knee arthroplasty, hip arthroplasty, spine surgery, and trauma surgery, as further described in the brochure “Acumen™ Surgical Navigation System, Surgical Navigation Applications” (2003), available from EBI, L.P. A more detailed description of an exemplary surgical navigation system is provided in James B. Stiehl et al., Navigation and Robotics in Total Joint and Spine Surgery, Chapter 1: Basics of Computer-Assisted Orthopedic Surgery (CAOS), Springer-Verlag (2004).

Computer 112 can be any computer capable of properly operating surgical navigation devices and software, such as a computer similar to a commercially available personal computer that comprises a processor 126, working memory 128, core surgical navigation utilities 130, an application program 132, stored images 134, and application data 136. Processor 126 is a processor of sufficient power for computer 112 to perform desired functions, such as one or more microprocessors. Working memory 128 is memory sufficient for computer 112 to perform desired functions such as solid-state memory, random-access memory, and the like. Core surgical navigation utilities 130 are the basic operating programs, and include image registration, image acquisition, location algorithms, orientation algorithms, virtual keypad, diagnostics, and the like. Application program 132 can be any program configured for a specific surgical navigation purpose, such as orthopedic application programs for unicondylar knee (“uni-knee”), total knee, hip, spine, trauma, intramedullary (“IM”) nail, and external fixator. Stored images 134 are those recorded during image acquisition using any of the imaging systems previously discussed. Application data 136 is data that is generated or used by application program 132, such as implant geometries, instrument geometries, surgical defaults, patient landmarks, and the like. Application data 136 can be pre-loaded in the software or input by the user during a surgical navigation procedure.

Output device 116 can be any device capable of creating an output useful for surgery, such as a visual output and an auditory output. The visual output device can be any device capable of creating a visual output useful for surgery, such as a two-dimensional image, a three-dimensional image, a holographic image, and the like. The visual output device can be a monitor for producing two and three-dimensional images, a projector for producing two and three-dimensional images, and indicator lights. The auditory output can be any device capable of creating an auditory output used for surgery, such as a speaker that can be used to provide a voice or tone output.

Still referring to FIG. 2, removable storage device 118 can be any device having a removable storage media that would allow downloading data, such as application data 136 and patient anatomical data 124. The removable storage device can be a read-write compact disc (CD) drive, a read-write digital video disc (DVD) drive, a flash solid-state memory port, a removable hard drive, a floppy disc drive, and the like.

Tracking system 120 can be any system that can determine the three-dimensional location of devices carrying or incorporating markers that serve as tracking indicia. An active tracking system has a collection of infrared light emitting diode (ILEDs) illuminators that surround the position sensor lenses to flood a measurement field of view with infrared light. A passive system incorporates retro-reflective markers that reflect infrared light back to the position sensor, and the system triangulates the real-time position (x, y, and z location) and orientation (rotation around x, y, and z axes) of an array 122 and reports the result to the computer system with an accuracy of about 0.35 mm Root Mean Squared (RMS). An example of a passive tracking system is a Polaris® Passive System and an example of a marker is the NDI Passive Spheres™, both available from Northern Digital Inc. Ontario, Canada. A hybrid tracking system can detect active and active wireless markers in addition to passive markers. Active marker based instruments enable automatic tool identification, program control of visible LEDs, and input via tool buttons. An example of a hybrid tracking system is the Polaris® Hybrid System, available from Northern Digital Inc. A marker can be a passive IR reflector, an active IR emitter, an electromagnetic marker, and an optical marker used with an optical camera.

Arrays 122 can be probe arrays, instrument arrays, reference arrays, calibrator arrays, and the like. Arrays 122 can have any number of markers, but typically have three or more markers to define real-time position (x, y, and z location) and orientation (rotation around x, y, and z axes). As will be explained in greater detail below, an array comprises a body and markers. The body comprises an area for spatial separation of markers. In some embodiments, there are at least two arms and some embodiments can have three arms, four arms, or more. The arms are typically arranged asymmetrically to facilitate specific array and marker identification by the tracking system. In other embodiments, such as a calibrator array, the body provides sufficient area for spatial separation of markers without the need for arms. Arrays can be disposable or non-disposable. Disposable arrays are typically manufactured from plastic and include installed markers. Non-disposable arrays are manufactured from a material that can be sterilized, such as aluminum, stainless steel, and the like. The markers are removable, so they can be removed before sterilization.

Planning and collecting patient anatomical data 124 is a process by which a clinician inputs into the surgical navigation system actual or approximate anatomical data. Anatomical data can be obtained through techniques such as anatomic painting, bone morphing, CT data input, and other inputs, such as ultrasound and fluoroscope and other imaging systems.

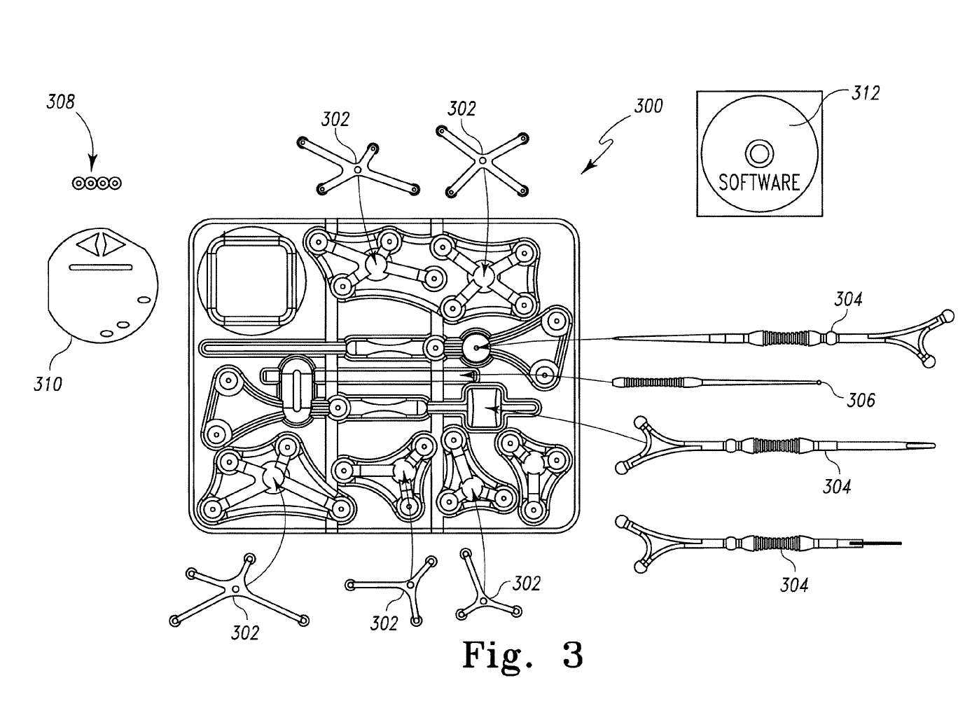

FIG. 3 shows orthopedic application kit 300, which is used in accordance with the present teachings. Application kit 300 is typically carried in a sterile bubble pack and is configured for a specific surgery. Exemplary kits can comprise one or more arrays 302, surgical probes 304, stylus 306, markers 308, virtual keypad template 310, and application program 312. Orthopedic application kits are available for unicondylar knee, total knee, total hip, spine, and external fixation from EBI, L.P.

In a total knee arthroplasty (TKA), a cutting guide such as those known in the art can be configured with an array such as array 302 and can thus be positioned relative to a bone using surgical navigation. In practice, however, the procedures can be difficult to implement. Although the required position of the cutting guide relative to the bone can be indicated on the screen of the navigation system, in practice it is extremely difficult to attach the guide in precisely the right position. The attachment procedure requires drilling holes through the bone into which bone screws are inserted to hold the guide in place. If the surgeon lets go of the cutting block before it is anchored, it will likely move and then must be repositioned. Once the holes are drilled, further adjustment of the position of the guide is often not possible. Exact matching of the position and orientation of the guide with the ideal position indicated on the screen is therefore extremely difficult.

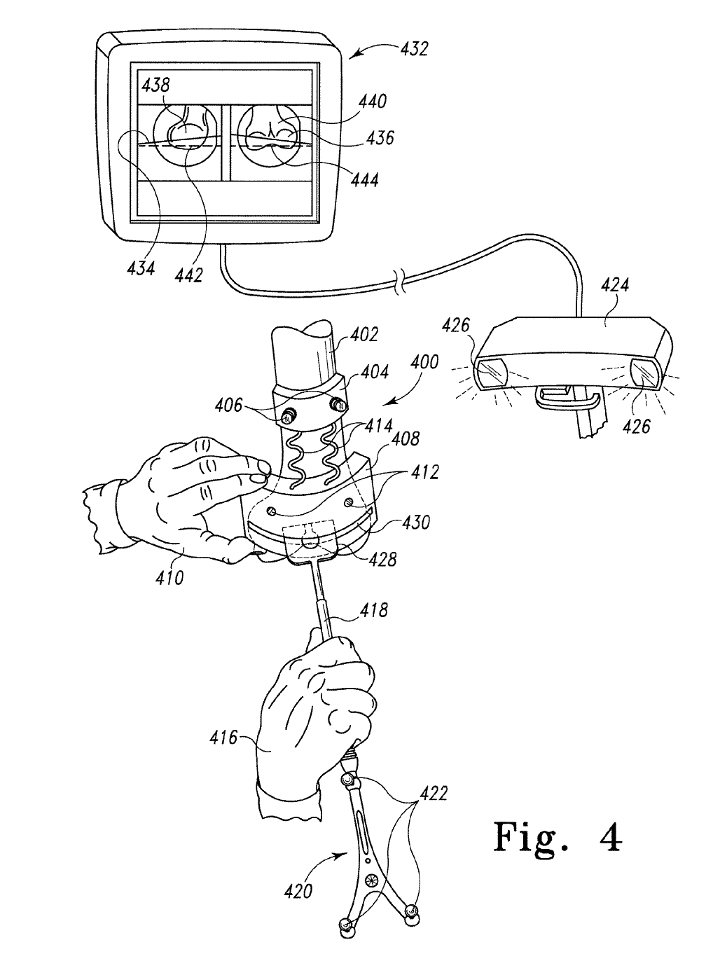

In accordance with the present teachings, FIG. 4 illustrates a cutting guide instrument 400 being used to assist a surgeon make a cut to femur 402. In this embodiment, the position of femur 402 is tracked using an array (not shown in FIG. 4) that is attached to the femur in accordance with the above teachings. Instrument 400 includes an anchoring block 404 which is shown secured to femur 402 by means of pins or nails 406 that extend through corresponding holes in block 404. Instrument 400 also includes a cutting guide or block 408, shown in FIG. 4 as being positioned by physician's hand 410. Cutting block 408 includes two cylindrical bores 412 through which pins (not shown in FIG. 4) can be inserted to secure cutting block 408 to femur 402 in a desired location. Anchoring block 404 and cutting block 408 can be formed of a wide variety of materials, including surgical stainless steel.

Two connecting members 414 connect the anchoring block 404 to the cutting block 408. In the illustrated embodiment the connecting members may be formed from plastic or rubber coated single strand wire, thereby providing “malleable” members that retain their shape when deformed. Depending upon the stiffness desired, one of skill in the art could select various gauges or thicknesses of wire. In the specific embodiment illustrated in FIG. 4, it is generally preferable that the wire or whatever structure is used for connection members 414 retain its shape after it has been deformed. For most materials selected for connection members 414, the extent to which the connection members maintain their shape (i.e., hold block 408 in position) is proportional to the resistance they provide against movement. The extent to which the connection members 414 maintain their shape once deformed is thus balanced against the commensurate resistance to movement as a design parameter. In any event, in the embodiment illustrated in FIG. 4, the malleable members 414 allow the physician to move cutting block 408 but provide sufficient resistance to movement so that the cutting block stays in place when the physician removes his hand 410 from it. This frees the physician's hand 410 to accomplish other tasks in the operating room. As described in more detail below, in other embodiments, the connecting members do not provide resistance to movement, but instead merely permit cutting block 408 to move relative to anchoring block 404 within a pre-determined range of motion and prevent movement of cutting block 408 relative to anchoring block 404 beyond the pre-determined range of motion.

Still referring to FIG. 4, physician's right hand 416 is shown holding spatula probe 418 having array 420 consisting of three reflective spheres 422. Spheres 422 of array 420 are detected by optical locator 424 having cameras 426. As discussed above, the surgical navigation system tracks the position of arrays such as array 420 and thereby also tracks the position of components that are connected to the arrays, such as spatula 428 of spatula probe 418. In FIG. 4, spatula 428 is shown aligned with cutting slot 430 of cutting block 408, i.e., spatula 428 is located in the same plane as slot 430. In this manner, monitor 432 of the surgical navigation system displays lines 434 and 436, which indicate the real time position of the spatula and thus slot 430 relative to side 438 and front 440 images of the tracked femur 402. Dashed lines 442 and 444 indicate the desired position of slot 430 of cutting block 408. At this point in the procedure, cutting block 408 still has not been secured.

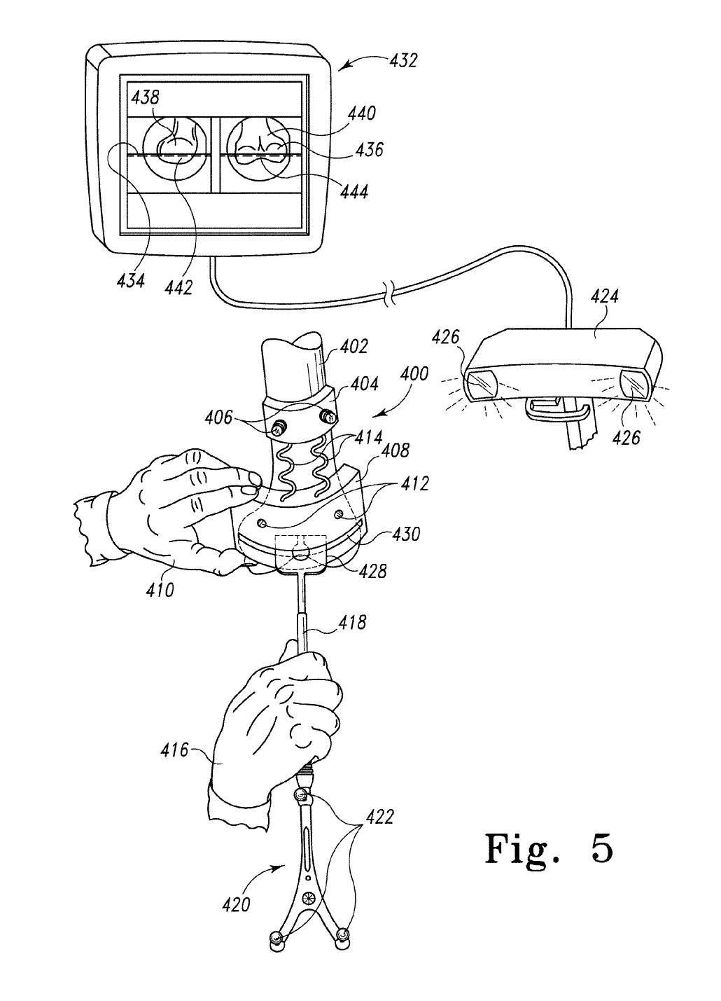

The physician moves cutting block 408 against the resistance of connecting members 414 until slot 430 is aligned in the desired plane relative to femur 402, as is indicated by the side and front views of the femur shown on monitor 432. As shown in FIG. 5, once cutting block 408 is aligned in the desired position, lines 434 and 436 align with and are essentially superimposed on lines 442 and 444, respectively, indicating that the cutting block is in the desired position. As discussed above, the physician may release his hand 410 from the cutting block 408, in order to, e.g., pick up pins and a fastening instrument to secure block 408. Meanwhile, connecting members 414 hold block 408.

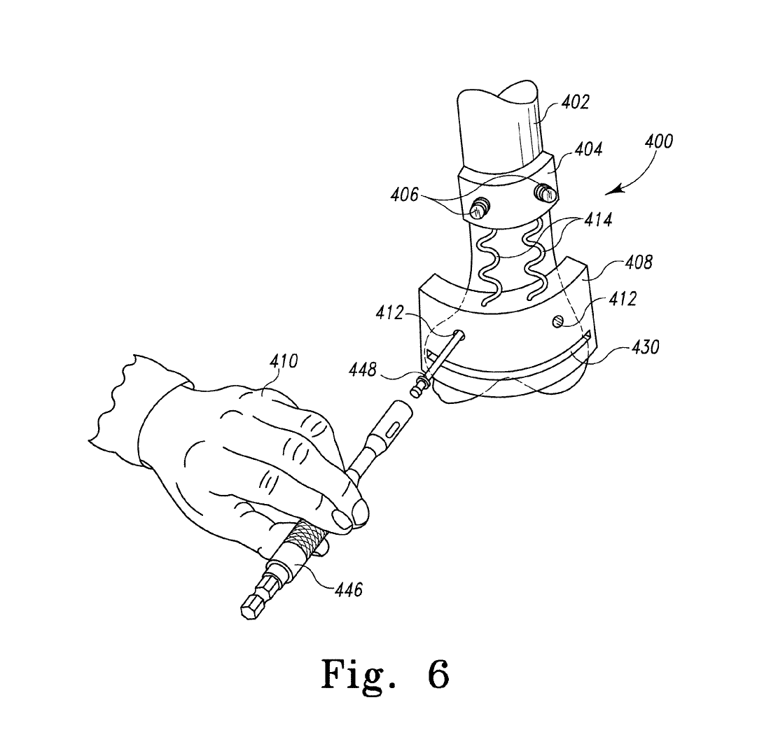

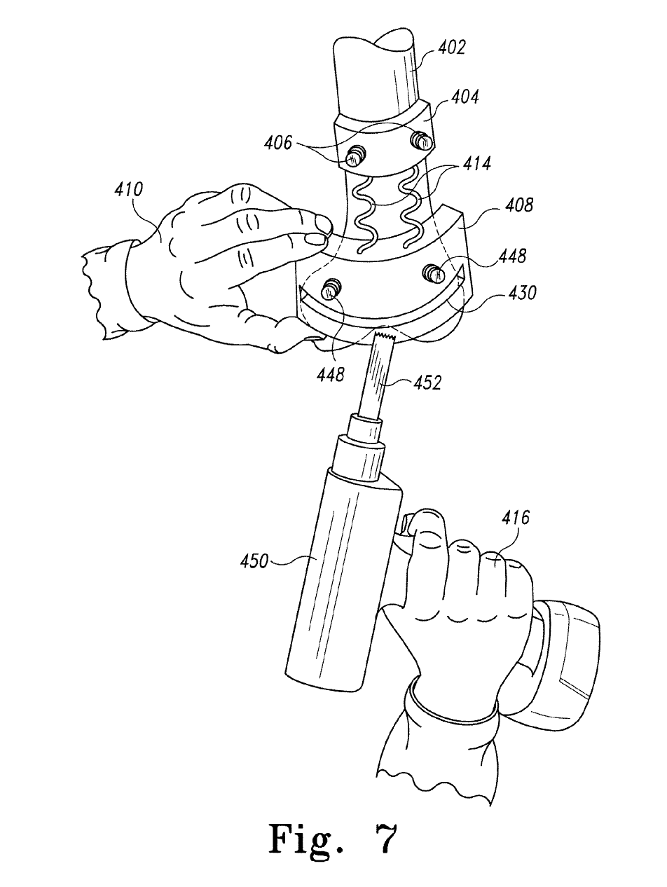

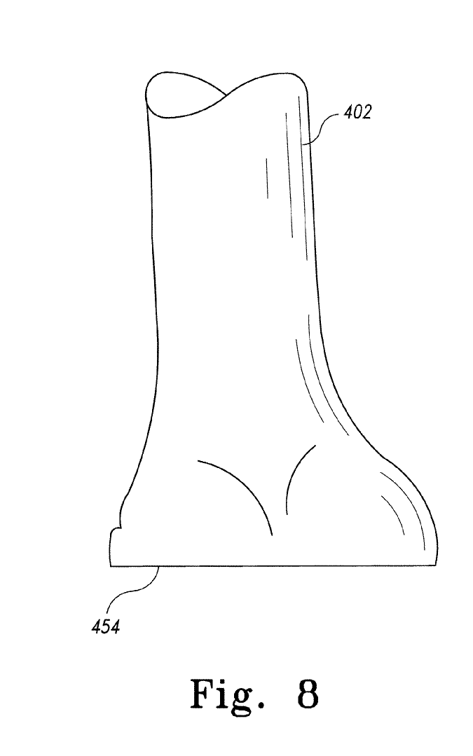

Next, as shown in FIG. 6, the physician's hand 410 holds a tapping instrument 446 to insert nails or pins 448 into holes 412 of block 408. A hammer or surgical mallet (not shown) is used to insert the surgical nails into bores 412 and the femur, as is known in the art. As shown in FIG. 7, once block 408 is secured to femur 402, the physician can use a surgical saw 450 having blade 452 to make the desired cut 454 to the femur 402, as shown in FIG. 8.

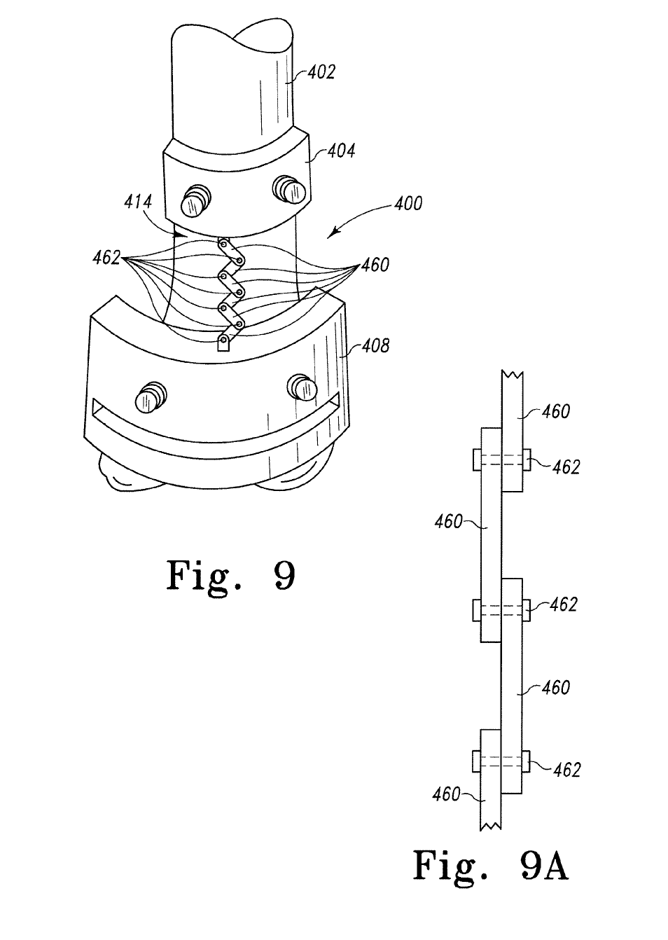

While the connecting members 414 are illustrated above as malleable members, one of skill in the art would readily recognize alternative embodiments for the connecting members in accordance with the present teachings. For example, FIG. 9 illustrates anchoring block 404 connected to cutting block 408 by means of a connecting member 414 having tabs 460 that are connected to one another by pins 462. With reference to FIG. 9A, pins 462 can be configured to press together two tabs 460 such that resistance is provided to movement of the connecting members relative to one another. Alternatively, in those embodiments in which the cutting block is merely to be maintained within a predetermined range of motion relative to anchoring block 404, a loose fit between tabs 460 can be arranged. In this event, the length of the connecting member can define the predetermined range of motion. It should also be understood that while the guiding surface of the cutting block 408 is shown as a cutting slot 430, the present teachings are not so limited. Generally, the guiding surface for the cutting instrument (and also the surface against which the spatula probe or other instrument is aligned) may or may not be enclosed by a slot configuration as illustrated herein. Some surgeons prefer a single flat surface which guides the cutting blade of the cutting instrument.

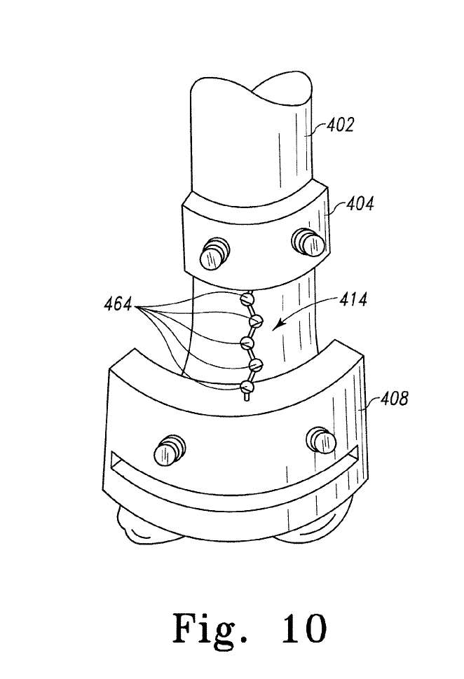

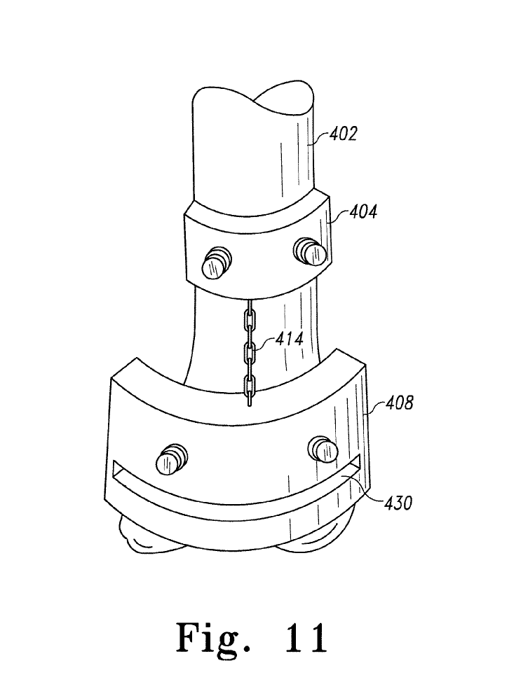

FIG. 10 illustrates a connecting member 414 that includes ball and socket connections 464, which can also be configured to provide resistance to movement. FIG. 11 illustrates a connecting member 414 in the form of a chain. The length of the chain defines the predetermined range of motion of the cutting block relative to the anchoring block. In view of these teachings, one of skill in the art would readily recognize further alterations to the connecting members that are within the spirit and scope of the appended claims.

While exemplary embodiments incorporating the principles of the present teachings have been disclosed hereinabove, the present teachings are not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An instrument for use with a surgical navigation system to aid in cutting a bone, comprising:
a first block configured for attachment to a bone;
a second block having a guiding surface and being configured for attachment to a bone; and
a connecting member connecting the first block to the second block, the connecting member permitting the second block to move relative to the first block within a pre-determined range of motion and preventing movement of the second block relative to the first block beyond the pre-determined range of motion.

2. The instrument of claim 1, wherein the connecting member provides resistance to movement of the second block relative to the first block, whereby the second block can maintain its position without a person holding it.

3. The instrument of claim 1, wherein the pre-determined range of motion is defined by the length of the connecting member.

4. The instrument of claim 1, wherein the second block comprises a distal femur cutting block.

5. The instrument of claim 1, wherein the second block comprises a tibial cutting block.

6. The instrument of claim 1, wherein the first block is configured for attachment to a femur.

7. The instrument of claim 1, wherein the connecting member retains its shape when deformed.

8. A method of cutting a bone during a surgery aided by a surgical navigation system, comprising:
providing a first block configured for attachment to a bone and a second block having a guiding surface and being configured for attachment to a bone;
connecting the first block to the second block with a connecting member such that the second block is permitted to move relative to the first block within a pre-determined range of motion but is prevented from moving relative to the first block beyond the pre-determined range of motion;
attaching the first block to a first bone of a patient;
using the surgical navigation system to position the second block in a desired cutting location;
attaching the second block to the first bone or the second bone; and
cutting the bone to which the second block has been attached.

9. The method of claim 8, wherein the positioning of the second block comprises placing a tool that is tracked by the surgical navigation system adjacent the guiding surface and aligning the guiding surface with a desired cutting location.

10. The method of claim 9, wherein the desired cutting location is displayed on a monitor.

11. The method of claim 10, wherein the location of the tracked tool is displayed on the monitor, whereby the real time position of the guiding surface may be compared to the desired cutting location on the monitor.

12. The method of claim 8, wherein the positioning of the second block comprises placing a spatula probe that is tracked by the surgical navigation system in the slot and aligning the guiding surface with a desired cutting location.

13. The method of claim 8, wherein the bone that is cut is a femur.

14. The method of claim 13, wherein the cut made is a distal femoral cut.

15. The method of claim 8, further comprising selecting a material for the connecting member that is flexible but provides resistance to movement.

16. The method of claim 15, further comprising removing all human contact from the second block and the second block thereafter maintaining its position.

17. The method of claim 8, wherein the positioning of the second block relative to the first bone or a second bone comprises positioning the second block relative to the first bone, whereby the method comprises attaching the first and second blocks to the same bone.

18. The method of claim 17, wherein the first bone comprises a femur.

19. The method of claim 8, wherein the step of connecting the first block to the second block with a connecting member comprises selecting a connecting member that retains its shape when deformed.
